(19) United States
(12) Patent Application Publication
(10) Pub. No.: US 2005/0182483 A1
(43) Pub. Date: Aug. 18, 2005

(54) Percutaneously placed prosthesis with thromboresistant valve portion

(75) Inventors: Thomas A. Osborne, Bloomington, IN (US); Brian C. Case, Bloomington, IN (US); Jacob A. Flagle, Bloomington, IN (US); Grant T. Hoffman, Bloomington, IN (US)

Correspondence Address: COOK GROUP PATENT OFFICE, P.O. BOX 2269, BLOOMINGTON, IN, 47402, US

(73) Assignee: Cook Incorporated, Bloomington, IN (US)

(21) Appl. No.: 11/056,903

(22) Filed: Feb. 11, 2005

Related U.S. Application Data
(60) Provisional application No. 60/543,753, filed on Feb. 11, 2004.

(51) Int. Cl. A61F 2/06; A61F 2/24

(52) U.S. Cl. 623/1.24; 623/2.38; 623/2.42

(57) ABSTRACT

A venous valve prosthesis having a substantially non-expandable, valve portion comprising a valve-closing mechanism, such as a pair of opposing leaflets; and an anchoring portion, such as one or more self-expanding frames or stents that are expandable to anchor the prosthesis at the implantation site. In one embodiment, the rigid valve portion includes a deposition of material such as pyrolitic carbon to reduce the thrombogenecity of the blood-contacting surfaces. The anchoring portions preferably include a covering, such as a tubular construct of synthetic or collagen-derived material (such as a bioremodelable ECM material), which attaches about the support structure such that blood flow is directed through the valve mechanism as it transitions from the larger diameter anchoring portion to the intermediate, smaller-diameter portion of the prosthesis. In another embodiment, the valve support housing and valve-closing elements are delivered in a collapsed, folded, and/or dissembled state sized for delivery, then manipulated in situ to the second expanded configured following deployment.

### CROSS-REFERENCE TO RELATED APPLICATIONS

This application claims priority of provisional application Ser. No. 60/543,753, filed Feb. 11, 2004.

### TECHNICAL FIELD

This invention relates to prosthetic valves percutaneously placed in the vascular system of mammals to augment or replace the function of the natural valves. This invention relates primarily to venous valves which would be percutaneously placed in the veins of the legs to replace the function of diseased or otherwise non-functioning venous valves.

### BACKGROUND OF THE INVENTION

Chronic venous insufficiency is essentially caused by venous hypertension and chronic venous stasis caused by valvular incompetence. As a result, the height of the blood column from the lower legs to the heart becomes longer, resulting in increased pressure in the veins of the legs. The resulting increase in pressure causes the veins to further dilate and the remaining valves to become incompetent. The disease progresses from varicose veins to ulcerations on the foot and lower leg which cannot heal due to the lack of adequate blood flow to and from the area.

The most common treatments for the disease consist of elevating the legs above the heart, to relieve the pressure in the veins and aid circulation back to the heart and pressure stockings, which help to constrict the veins and retard the expansion due to the increased pressure. These treatments only serve to slow the progress of the disease. In addition, these treatments can greatly interfere with normal daily activity. The ideal solution would be a minimally invasive, blood compatible, permanent prosthetic device that will replace the function of the valves. Many prosthetic valve devices have been invented for the purpose of restoring proper blood flow. One such device is disclosed in U.S. Pat. No. 6,315,793. This device is a mechanical “check” valve that is surgically implanted in the veins of the patient. Although this device has the ability to restore correct flow, it must be surgically placed. Since blood flow is poor at best in these patients, surgery can be very traumatic and require extended, problematic recovery. One percutaneously placeable valve is described in U.S. Pat. No. 5,397,351. This is a ball and cage device designed so that it can be collapsed and placed through a catheter type introducing system. Due to the complex structure of this device, it is prone to forming clots which could interfere with its function and possibly result in emboli being generated which could flow back through the heart, out into the lungs and become a dangerous pulmonary embolism. Patients using this type of device would be required to take anti blood clotting drugs for the rest of their lives. Another percutaneous valve device is described in U.S. Pat. No. 6,200,336. This device is a flexible “flap” type valve that is mounted in an expandable wire frame. When this device is deployed, the dimensions of the frame are controlled by the dimensions of the vein. As a result, the final shape and dimension of the frame might be too loose or too tight to allow the flap valve to operate effectively. Another percutaneously placed valve system is described in U.S. Pat. No. 6,299,637. This device is another type of check valve that uses an expandable, covered wire frame valve. The valve is carried in and mounted inside an expandable “Z” type stent. This device has the same problem as the previously mentioned device in that it must expand to a specific size in order to be effective.

Veins, by their nature, do not have a set size or shape. They can expand and contract depending on whether the patient is at rest, lying down or vertical and active. In addition, the valve system of the above mentioned patent would be prone to clot formation and would require that the patient be on anti clotting drugs. Antithrombogenic surface treatments have been used in surgically implantable heart valves, but these devices are necessarily rigid and non-expandable and thus, are not suitable for intravascular delivery or implantation in the peripheral venous system, such as the lower legs to treat chronic venous insufficiency. Therefore, what is needed is an artificial venous valve comprising a thromboresistant material and which includes an expandable portion to anchor the non-expandable valve mechanism portion in a manner that directs or permits antegrade flow through the valve, while restricting retrograde flow.

### SUMMARY OF THE INVENTION

The foregoing problem is solved by the present prosthetic valve system comprising a valve mechanism having a substantially fixed-diameter support structure or frame of a first diameter sized for intravascular delivery and one or more expandable stents or other anchoring support structures attached to or integral with the valve mechanism frame, preferably located at both ends of the device. The valve support housing or body would be sized so as to fit inside a delivery sheath that could be introduced into the vein by percutaneous (Seldinger) technique. The expandable portions would be sized so that in the collapsed condition (e.g., to the first diameter), the prosthesis would fit inside the delivery sheath. When the system is deployed or expelled from the delivery sheath inside a vessel, the stents would expand to the vein inside diameter and anchor the valve securely in the vein. In a preferred embodiment, the anchoring portions would be covered, at least along the portion interconnecting the expandable stents and the valve portions (housing), so that blood flow would be directed through the valve and not be allowed to be flow therearound. The covering would also serve to prevent or limit the reflux of blood back around the valve during back flow or negative pressure conditions. Alternatively, the covering could be configured to allow a controlled amount of reflux to prevent pooling of blood adjacent the valve-closing elements. In other embodiments, the prosthesis may be configured such that the vessel adheres to the support structure and seals itself against passage of blood through the transitional areas between the fully expanded support structure and the smaller-diameter valve portion.

Preferably, the blood-contacting surfaces of the actual valve parts (e.g. frame, leaflets, etc.) include a smooth layer or coating of material that inhibits the formation of blood clots which could migrate to the heart and lungs or perhaps interfere with valve function. A preferred material is pure pyrolitic carbon which has been shown to be very thromboresistant, as disclosed in U.S. Pat. No. 6,410,087 (column 1, line 37). The process for depositing pyrolitic carbon on a medical prosthesis is described in the '087 patent, the entire disclosure of which is hereby incorporated by reference. The pyrocarbon described has the characteristics of a relatively high density of at least about 1.5 gm/cubic centimeter, an apparent crystallite size of about 200 angstroms or less and high isotropy. This material has been shown to be very inert and thromboresistant and has become the material of choice for surgically placed heart valves. Because depositing carbon on the valve components results in a valve and support mechanism that is substantially rigid and non-flexible (typically), it cannot be readily compressed or collapsed down for delivery. Therefore, the valve mechanism needs only to be sufficiently small for intravascular delivery, while other support structure, such as stents located at each end, provides the anchoring function such that the valve mechanism does not migrate within the vessel.

In a second aspect of the invention, the valve portion of the support structure includes a pre-deployment configuration having a smaller overall diameter for delivery such that after deployment within the vessel, the valve support housing and valve-closing elements are unfolded and/or assembled at the implantation site to produce a functioning valve having second diameter larger than that which could be accommodated by the delivery system. In one embodiment the semi-collapsed valve support housing is unfolded and locked into a tubular configuration, whereby the one or more leaflets or other valve-closing elements are similarly unfolded from the delivery and inserted into place within the valve support housing, such as with the aid of radiopaque marker to produce a functioning valve. The leaflets can comprise a fan or hinged configuration which is unfolded upon deployment and inserted into apertures or other appropriately configured structure that engages the leaflet and allows it to pivot other otherwise function to selectively restrict retrograde blood flow. In a second embodiment, the valve support housing and/or valve-closing elements are each delivered as multiple components that are assembled at the implantation site. For example a leaflet could comprise a first and second half with interlocking edges and/or magnets, etc., that allow the leaflet to securely fit together once the housing has assumed its final configuration such that the assembled leaflet could be inserted in place. The valve-closing elements, valve support housing, and other substantially non-compressible can be assembled using intravascular instrumentation, connecting wires, or other means to assemble the components following deployment.

### BRIEF DESCRIPTION OF THE DRAWING

Embodiments of the present invention will now be described by way of example with reference to the accompanying drawings, in which:

FIG. 1 depicts a cross-sectional side view of a bileaflet embodiment of the present invention;

FIG. 2 depicts a cross-sectional view of an bileaflet embodiment of the present invention in which the anchoring portions each comprise a covering to direct blood flow through the valve portion;

FIG. 3 depicts a partially sectioned side view of a valve portion comprising a single leaflet;

FIG. 4 depicts an cross-sectional view of the embodiment of FIG. 1 collapsed inside a delivery member;

FIG. 5 depicts a cross-sectional view of an embodiment of the present invention in which the valve portion is disposed within the anchoring portion;

FIG. 6 depicts a cross-sectional view of an embodiment of the present invention in which the valve mechanism comprising ball valve and seating elements;

FIG. 7 depicts a perspective view of an alternative single-leaflet embodiment of the present invention;

FIGS. 8-9 depict perspective views of the present invention of a valve support housing embodiment of the present invention which is expandable to a second configuration using a balloon;

FIG. 10 depicts an detail end view of an alternative embodiment in which the seam edges of the valve mechanism housing engage and lock with one another;

FIGS. 11-12 depict end views of an embodiment of the present invention which is balloon-expandable from a first to a second configuration and includes a locking seam;

FIGS. 13-14 depicts side views of an expandable leaflet embodiment having a fan configuration;

FIG. 15 depicts a perspective view of an expandable leaflet embodiment which is unfoldable into a second configuration;

FIG. 16 depicts a cross-sectional view of a embodiment of valve support housing having radiopaque markers to locate the leaflet engagement points thereon;

FIG. 17 depicts a partially sectioned view of the embodiment of FIG. 15 being manipulated into position using an elongate member and a retention wire;

FIG. 18 depicts a side view of a leaflet embodiment having first and second portions that are assembled following deployment of the valve support housing;

FIG. 19 depicts a side view of a support structure formed from a single piece of tubing of a superelastic material.

FIG. 20 depicts a side view of the embodiment of FIG. 19 in which the anchoring portions are formed into an expanded configuration;

FIG. 21 depicts a partially sectioned side view of an embodiment of the present invention in which the valve-closing element comprises an umbrella configuration;

FIG. 22 depicts a partially sectioned perspective view of a prosthesis in which the ring-like valve support housing comprises a plurality of assembleable components.

FIG. 23 depicts a detail view of an embodiment of connectors between section of the valve support frame of FIG. 21.

### DETAILED DESCRIPTION

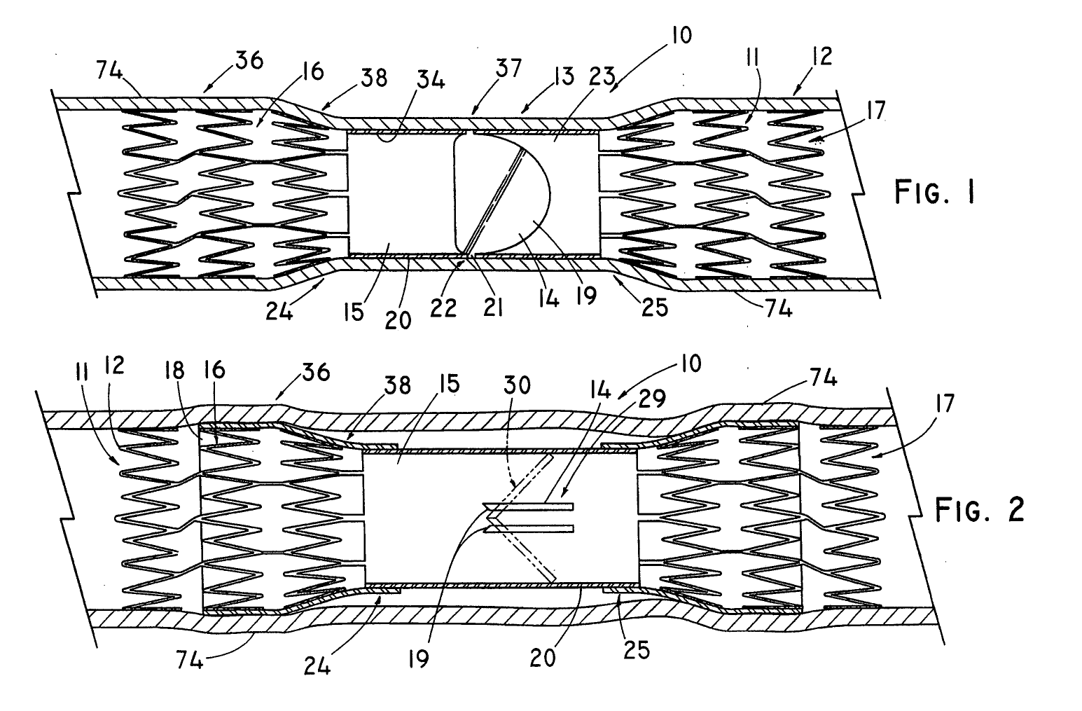

FIGS. 1-23 depicted a series of embodiments of the present invention of an implantable prosthesis 10, such as an artificial venous or heart valve that includes a support structure 11 comprising one or more anchoring portions 12 configured for engaging the vascular wall, and a valve portion 13 comprising a valve-closing mechanism 14, such as one or more sealing elements, and a valve support housing 15 that comprises a substantially rigid material such that the valve portion 13 or valve support housing 15 has limited collapsibility and thus, relies on the anchoring portion(s) 12 to expand and fully engage the vessel wall to anchor the prosthesis 10 thereagainst. Barbs (not shown) may further serve to help anchor the prosthesis at the implantation site. The valve portion 13 includes either an outer diameter sufficiently small for delivery through a delivery member 27, whereby the anchoring portion provide the necessary radial expansion to engage the vessel; or the valve portion 13 is configured to have limited expandability (such as by using a non-compliant balloon), be unfolded, or assembled in situ from two or more components following deployment from the delivery member 27.

FIG. 1 depicts a cross-sectional view of an illustrative prosthesis 10 of the present invention in which the support structure 11 comprises a valve portion 13 that includes a rigid tubular valve support housing 15 in which the valve-closing elements 14 comprise a pair of leaflets 19 or vanes that are configured to close against one another and provide a seal therearound to at least substantially prevent the reflux of blood back through the valve. The valve support housing 15 comprises a substantially rigid, non-collapsible element which is preferably comprises a antithombogenic surface or coating 34 that decreases resistance to blood flowing thereagainst and substantially reduces the ability of proteins, such as fibrin to attach to the blood-contacting surfaces 23 of the valve. An example of such a coating 34 includes a deposition of amorphous or pyrolitic carbon upon a suitable base material for forming a support structure, such as titanium, stainless steel, nitinol, Elgiloy, NP35N, ceramic, etc., that is sufficiently rigid to support the valve mechanism. If depositions such as pyrolitic carbon are used, the ability of the valve portion to flex or be made semi-collapsible can be limited. Therefore, the valve portion 13 typically comprises the smallest practical diameter for delivery through a delivery member 27 or sheath, an example of which is shown in FIG. 4. In the illustrative example, a pusher member 28 is used to urge the prosthesis 10 from the delivery sheath 27, however, any suitable apparatus for delivering a expandable prosthesis may be used. As for sizing of the prosthesis 10, a valve portion with an outside diameter of about 8 mm could be used in a vein with a lumen size or inside diameter of 12 mm. This would allow a delivery sheath with an outside diameter of about 9.0 mm (26.5 Fr). The delivery sheath 27 would have a wall thickness of about 0.010 to 0.015″, resulting in adequate clearance between the outside diameter of the valve body and the inside of the delivery sheath. Smaller valve portions 13 can be used by increasing the amount of flaring of the anchoring support structures 16,17 from the valve portion 13 such that the angles of the transitional areas 38 therebetween are increased.

Because the valve portion 13 is not configured to be self-expandable of a degree sufficient to anchor the prosthesis 10 in the vessel or other bodily lumen, one or more anchoring portions 12, such as self-expanding stents (e.g., the illustrative stainless steel or nitinol serpentine or zig-zag stents) can be attached about the valve portion which are expandable from a first diameter or configuration 35 within the delivery system (generally that of the valve portion 13) to a second diameter or configuration 36 that is sized to expand against the walls of the vessel to anchor the prosthesis therein. The scope of the invention also includes using a balloon delivery catheter over which the valve would be mounted having a single or multiple balloons to expand the anchoring portions to the second diameter or seat the anchoring portion (particularly if necessary to help embed optional anchoring barbs located on the frame). In the illustrative example, the anchoring portion 12 comprises a first anchoring support structure 16 attached about the first end 24 of the valve portion 13 and a second anchoring support structure 17 attached about the second end 25 of the valve portion 13. The anchoring support structure 16,17, which each comprises a series of interconnected serpentine stents that flare outward from the support housing ends 24,25 to which they are attached, can be soldered, sutured, or glued to the valve portion. They also may lock into or otherwise engage or attach to the valve portion as separate components. Alternatively, the anchoring portion 13 (anchoring support structures 16,17) may be integrally formed with the valve portion, such as being cut from a common piece of metal cannula. FIGS. 19-20 depict a section of nitinol tubing that is laser cut into the first configuration 35 wherein the anchoring support structure 16,17 comprise the same diameter as the valve support housing 15. The anchoring portions 13 are then formed by heat setting or cold working the first and second anchoring structures 16,17 such that they assume the second, expanded configuration 36. The prosthesis 10 is then compressed into a delivery system to reassume the first configuration 35, whereby it self-expands following deployment to anchor the prosthesis at the implantation site.

Referring now to both FIGS. 1 and 2, the illustrative pair of pivoting leaflets 19 which comprise the valve closing mechanism 14 are mounted to the valve support housing 15 by an attachment mechanism 37 which comprises a pair of first rotational elements or projections 21 disposed at either end of each leaflet 19, and which are sized to fit into a pair of second rotational elements 22 which comprise apertures or recesses formed in the inner walls of the valve support housing 15. Conversely, the first rotational elements 21 of the leaflets may comprise recesses while the corresponding projections comprising the second rotational element 22 are mounted on the inner surfaces of the valve support housing 15. One may contemplate alternative structures that permit the leaflets 19 to pivot about an axis from an open position 29 to a closed position 30 in order to selectively allow or restrict fluid flowing through the valve portion 13. While the illustrative embodiment of FIGS. 1 and 2 depict an embodiment having two coapting or contacting leaflets 19 which in the open position 29, define an orifice therebetween to allow blood flow, single leaflet embodiments also fall within the scope of the present invention, as do those embodiments having three or more valve-closing elements. In FIG. 3, a single valve-closing element 14 or leaflet 19 is positioned within the passageway 39 of the valve portion 13 in which the pivoting attachment mechanism 37 comprising the first and second rotational elements 21,22 is located centrally along the lateral edges 40 of each leaflet 19. The antegrade flow 41 forces the leaflet 19 to pivot and open, while retrograde flow causes the leaflet to return to the closed position and form a seal with the valve support housing 15. Preferably, the leaflet 19 is configured to fit against the inner surface of the support housing 15 such that is can only open in one direction. To help ensure timely closing of the valve to prevent retrograde flow 42 therethrough, the leaflet 19 can be configured or weighted such that automatically closes when antegrade flow 41 is not longer pushing the valve into the open position 29. A biasing means, such as spring wire, may also be used to assist in returning the valve to its closed state. In a related embodiment depicted in FIG. 7 the attachment mechanism 37 for the single valve-closing element 14 comprises a hinge 43 located at the base 44 of the valve-closing element 14. Otherwise, the valve 13 opens and closes in a similar manner to the embodiment of FIG. 3.

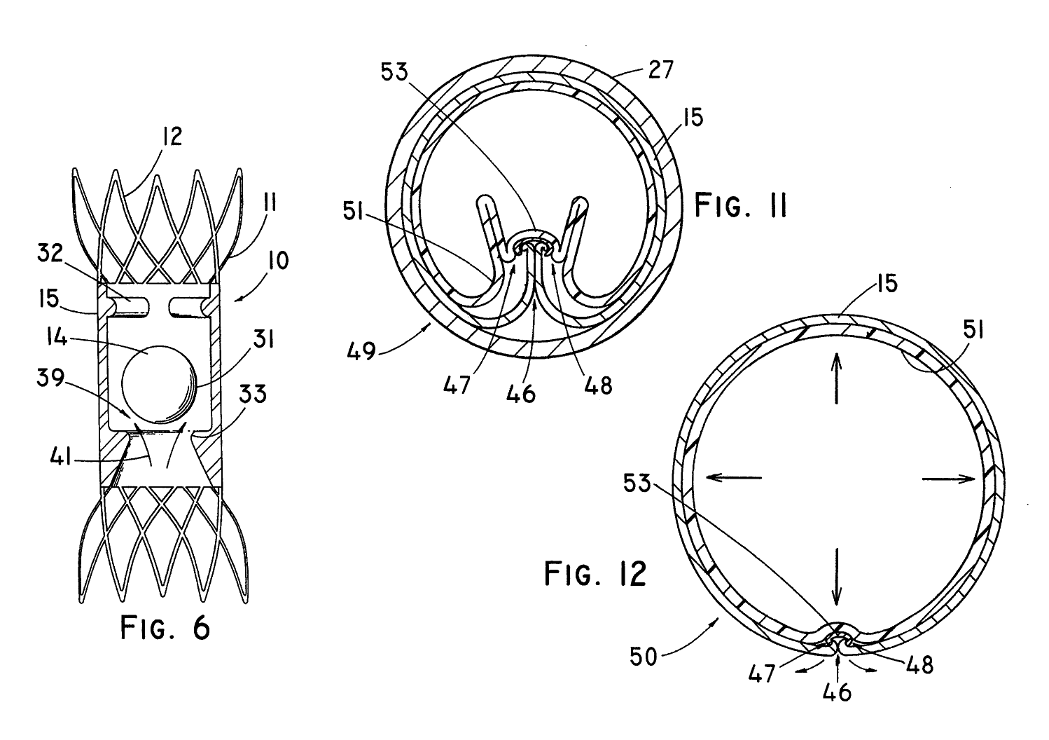

Another valve portion 13 embodiment is depicted in FIG. 6 in which the valve mechanism/valve closing element 14 comprises a ball valve 31 which is sized and weighted to be forceable upward by antegrade flow 41, which flows therearound until the cessation thereof causes the ball valve 31 to fall back down and seal against the seating ring 33 located distal thereto. Proximal retaining structure 32, such as the illustrative c-ring, is formed with or attached about the inner passageway 39 to limit the proximal movement of the ball valve 31 to prevent it from migrating out of the valve support housing 15. Any suitable structure to prevent passage of the ball valve 31 therepast, such as projections, narrowing of the passageway, etc., may be used. The illustrative embodiments are merely exemplary in nature and as such, the selection and configuration of the valve-closing mechanism of the valve portion 13 is not particularly critical to the understanding of the invention. Other designs, such as duck bill valves, are also contemplated for use in the present invention.

The embodiment of FIG. 2 depicts a covering 18 or sleeve of material that is configured to direct antegrade and retrograde flow into the valve portion and prevent it from leaking through the open cells within the support structure. The covering 18 can be made of naturally-derived biologic or collagen-based material, such as small intestinal submucosa (SIS), SIS and other extracellular collagen matrix materials (ECM) having the advantage of being able to bioremodel and endothelialize over time. SIS is commercially available from Cook Biotech, Inc., West Lafayette, Ind. Methods for preparing the SIS material are disclosed in a number of U.S. patents, such as U.S. Pat. Nos. 6,206,931, 6,358,284, and 6,666,892, the disclosures of each are hereby incorporated by reference into this application. ECM (SIS) material has the advantage of being able to remodel to vascular tissue (e.g., endothelium) after a period of time, typically within a month. This feature would further enhance the resistance to clot formation. ECM materials have an advantage over fixed collagen materials in that they are strengthened and no longer susceptible to degradation after they have remodeled. The bioremodelable covering can comprise a single ECM sheet formed into a tubular construct, multiple laminated sheets of ECM, a comminuted ECM and binder material ‘cast’ into a construct around the stent, a ‘sandwich’ of ECM layers over a stent (or visa versa), a hybrid synthetic-ECM multilaminate, or any other structure, formulation, or combination suitable to function as a graft prosthesis to funnel or direct blood through the prosthesis that would otherwise leak through the transitional, funnel-shaped area between the larger anchoring stent and the smaller valve-containing portion.

In addition to SIS and other bioremodelable coverings, cross-linked, non-remodelable collagen materials may be used as well. Alternatively, a synthetic biocompatible fabric, polymer, or other such material may be sewn, applied, or otherwise attached to the anchoring portion 12 of the support structure 11 using a standard technique appropriate for that particular material (e.g., sewing, heat welding, crimping, gluing, spraying, dipping, etc.). Examples of possible synthetic covering 18 include, but are not limited to polyester fiber (DACRON), ePTFE, silicone, polyurethane, and silk. The covering material 18 may be impregnated or coated with one or more pharmacological agents and elution controlling polymer layers or carriers, growth factors, seeded cells or genes, surface modifying agents for preventing adhesion of cells or proteins, and/or other bioactive agents. Drugs or other substances may be added to inhibit thrombus formation on the adluminal covering surface, reduce inflammation/hyperplasia at the implantation site, encourage encapsulation of the stent and/or encourage formation of an intimal layer, etc. In addition, the outer or abluminal surface of the covering 18 may be made porous or otherwise modified (e.g., include knurling or a suitable nanosurface) to encourage tissue ingrowth or cell adhesion to help anchor the prosthesis.

While the fluid-directing covering 18 of FIG. 2 advantageously restricts blood or fluid from flowing around the outside of the valve portion 13, it is within the scope of the invention for the anchoring portions 13 to include a partial covering (such as to allow limited reflux of blood) or lack any covering, such as the embodiment shown in FIG. 1. Because veins are generally very pliant, the transitional areas 38 where the anchoring support structures 16,17 are reduced in diameter to connect with the valve portion 13 can be sealed by the vessel 74 itself under certain conditions, especially if the outer surface 20 of the support structure 11 is modified to encourage permanent adhesion to the vessel over time. This can be accomplished by the use of a porous materials, materials with a high surface area, agents to increase adhesion or ingrowth, microstructure to engage and attach to the vessel wall 74 (e.g., microbarbs), or other well-known modifications that would cause the support structure to adhere to the wall and substantially reduce the opportunity for blood to seep through the transitional areas 38 or other locations along the implantation site.

The embodiments depicted in the figures discussed above include an anchoring portion 13 that includes a first and a second anchoring support structure 16,17 attached at either end of the valve portion 13. While this arrangement advantageously allows for a covering about both ends 24,25 of the valve portion 13 to direct blood through the valve, as well as providing a centering function with the vessel, it is within the scope of the invention to include a single anchoring support structure 16, such as one located only at the proximal end, thereby allowing the valve portion to extend distally therefrom, otherwise unsupported.

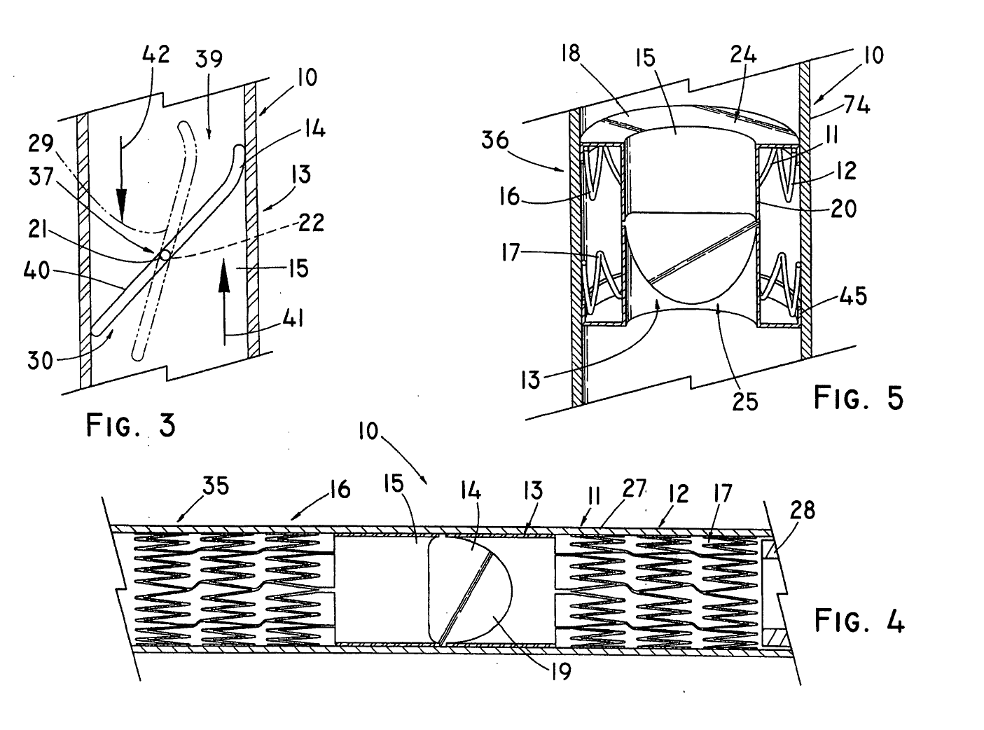

In another embodiment, the anchoring support structure 12 may be disposed external to or radially outward from the valve portions, such as depicted in the embodiment of FIG. 5. In the illustrative embodiment, a first anchoring support structure 16 is located about the first end 24 of the valve portion 13, where it is attached thereto via a bridge of covering 18, configured in a doughnut shape with cuff 45 that folds over the outside of the prosthesis 10 and its stitched or otherwise attached to the struts or bends of the anchoring support structure 16. Likewise, a second anchoring support structure 17 is attached about the second end 25 of the valve portion 13 in similar manner, such that when the prosthesis 10 is deployed, the anchoring portion 13 expands to engage the vessel while the valve portion, which is attached to the anchoring portion 12 by the unfolding covering 18, is centered therewithin, Alternatively, a single anchoring support frame 16 can be used that extends the length of the valve portion 13 and is attached to the covering 18 at both ends. In another alternative embodiment a single covering 18 extends the length of the valve portion 18, essentially assuming a drum-like configuration, which is then attached to the or more anchoring support structures 16,17. As an alternative to having the interconnection between the anchoring support structure and valve portion comprised only of the covering, a series of interconnecting arms (not shown) can be provided to extend outward from valve support housing 15 during radial expansion of the anchoring support structure 16,17 and attach to bends and/or struts thereof as an additional means of attachment.

While the present invention addresses the problem of anchoring a rigid, non-expandable prosthesis, such as the illustrative valve embodiments, within a vessel and still being able to deliver the prosthesis percutaneously, clinical barriers may exist to delivering such a large prosthesis to certain locations within the body, particularly if the access vessel is small, the pathway is tortuous, or the heart must be traversed. To further downsize the valve portion for delivery through a smaller delivery system, the valve support housing and valve-closing elements can be configured to have limited expandability/collapsibility to assume a larger shape upon deployment at the implantation site. This may be done is a number of ways, including unrolling, unfolding, assembling, or otherwise expanding the components of the valve portion into a functioning valve mechanism of a larger diameter than could otherwise be delivered through the optimally sized delivery system.

FIGS. 8-9 depict an embodiment of a valve portion 13 in which the valve support housing 15 include a open seam 46, whereby a first edge 47 defining the seam is folded under the second edge 48 so that the support housing 15 is basically rolled and partially compressed to reduce its overall diameter for delivery through a smaller delivery sheath. The degree of flexibility of the support housing and its ability to be rolled and compressed is largely determined by the rigidity and thickness of the material and its ability to flex without damaging any coatings or materials deposited thereupon. In the illustrative embodiment, a non-compliant balloon 51 (e.g., PET or nylon) is used to expand the valve support housing 15 from it first, compressed configuration 49 to a second, expanded configuration 50 for deployment. As the support housing 15 reaches the final diameter 50, structural adaptations 52 located near the first and second edges 47,48 allow the support housing 15 to lock in the expanded, second configuration 50. In the illustrative embodiment of FIG. 9, the locking structure 52 comprises a series of recesses and corresponding raised projections that slide and lock thereinto when the support housing 15 is sufficiently expanded. In an alternative embodiment depicted in FIG. 10, the first and second edges 47,48 are configured to lock end to end when the first edge 47 slides past the second edge 48 during expansion of the support housing 15. The illustrative configurations of the first and second edges 47,48 are merely exemplary of second edge 48 configurations adapted to capture the first edge 47 as the two edges come into end-to-end contact with one. One skilled in the art should be able to select other configurations that would function in a similar manner.

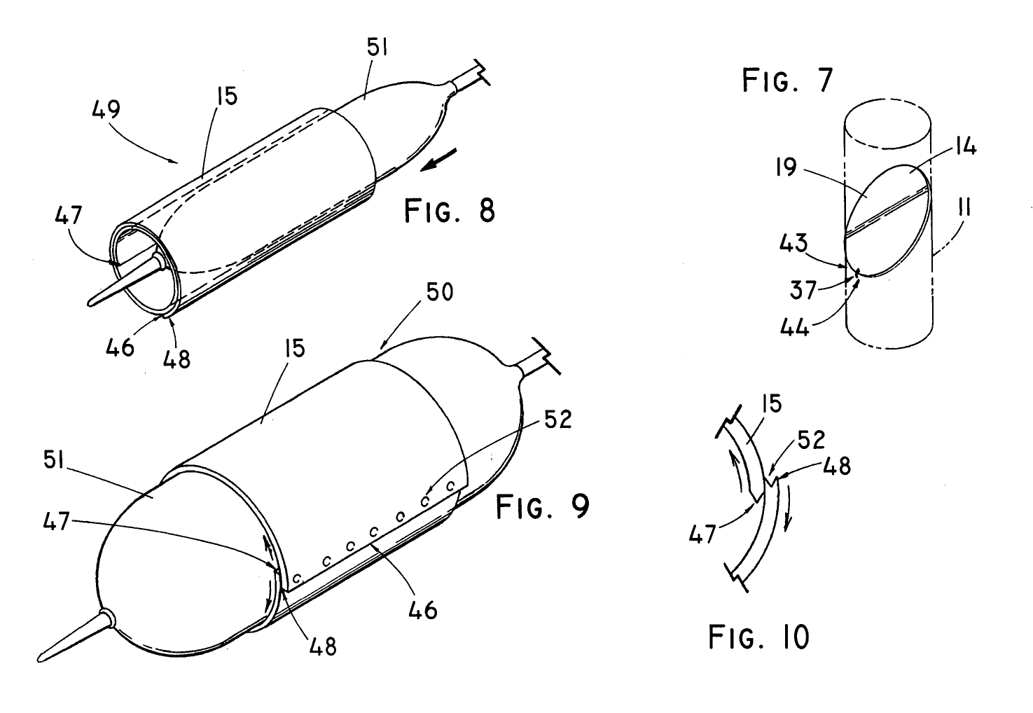

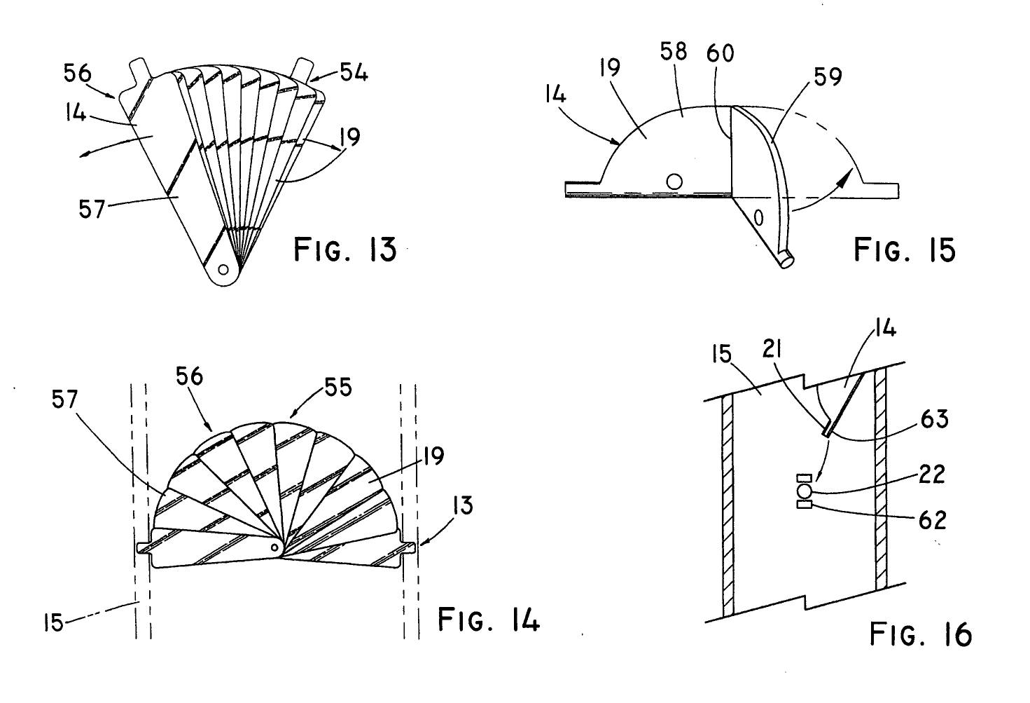

A second embodiment of a valve portion 13 with an expandable valve support housing 15 is depicted in FIGS. 11-12. The first and second edges 47,48 of the valve support housing, which are joined by a locking seam member 53 (a elongate member or series of shorter members), are folded inward to reduce the outside diameter of the housing in the compressed first configuration 49. As the valve support housing 15 is expanded by the balloon 27 to assume the expanded second configuration 50, the inverted edges 47,48 unfold outward such that support housing assumes a round configuration. At that point, the edges 47,48 defining the seam 46 are locking into place by the locking seam element 53, which is configured to allow rotational movement of the edges relative to one another until the support housing 15 is expanded to it intended final diameter. Once locked in place by the locking seam element 53, the edges 47,48 are held in position and the shape of the valve housing 15 maintained even after deflation of the dilation balloon 27.

Once an expandable valve support housing 15, such as those depicted in FIGS. 8-12, is expanded and locked into place, valve-closing elements can be inserted thereinto to produce a functioning valve portion 13. For example, leaflets can be configured to be deliverable through the same sheath used to deliver the valve support housing. The leaflets are then attached under fluoroscopy, such as by the use of instruments (e.g., grasping forceps) designed to manipulate the leaflets until they can engage the housing 15 to form an attachment mechanism 37 (e.g., inserting the pivoting projections 21 or tabs of the leaflet 19 into corresponding recesses or apertures 22 in the housing 15 as depicted in FIG. 1).

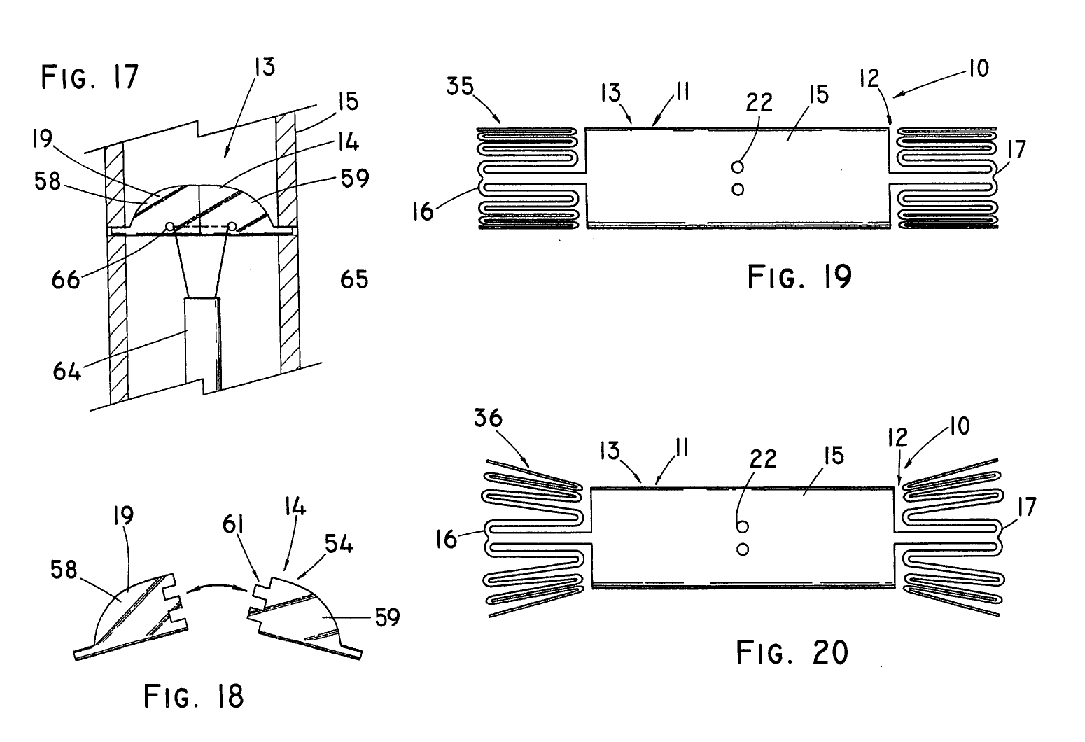

Like the housing, leaflets 19 or other valve-closing elements 14 that are not dimensioned for transcatheter delivery can either be rolled slightly like the valve support housing 15 of FIG. 8, if sufficiently flexible. For more rigid valve-closing elements 14 or leaflets, they can adapted to have a folded or collapsed configuration 54 and an expanded configuration 55 for placement in the valve support housing 15, such as the fan configuration 56 leaflet 19 of FIGS. 13-14. The illustrative leaflet 19 comprises a plurality of interconnect plates 57 which are configured to fold under one another like a fan in the first configuration 54 for delivery through the sheath, whereby the leaflet 19 is expanded for engagement with the valve support housing 15 to form a functioning valve portion 13. A second folded leaflet 14 embodiment is depicted in FIG. 15 which comprises a first leaflet portion 58 that is folded against a second leaflet portion 59 along a seam 60 which can comprise a hinge or bridge made of a flexible material such as a strip of polymer, fabric, thin metal, etc. The leaflet 19 is configured such that when folded, its diameter is reduced at least in one direction such that it can be delivered through a sheath smaller than would otherwise be possible.

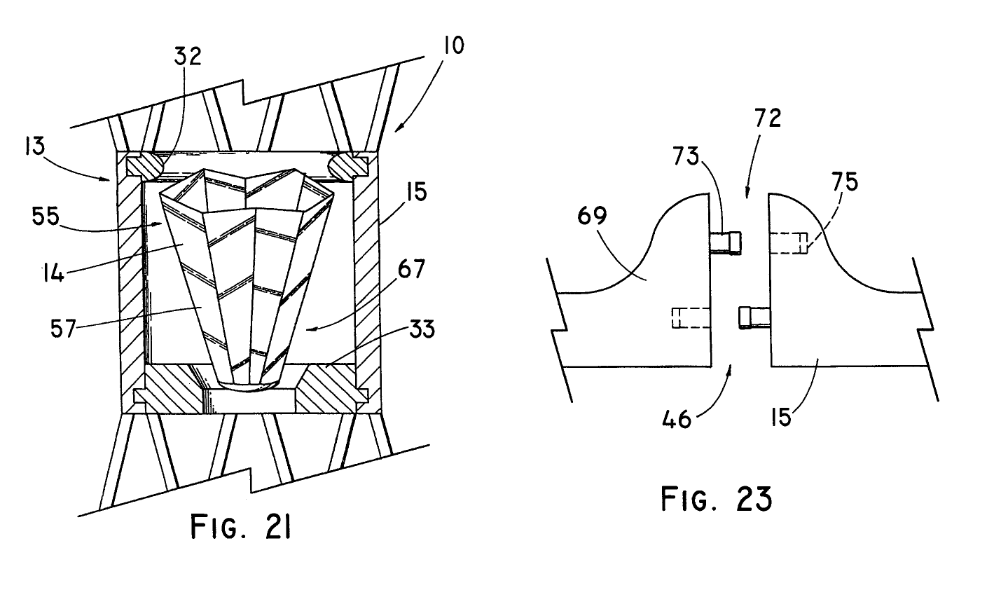

Another method of delivering a valve-closing element 14 in a first configuration 54 through a smaller sheath is depicted in FIG. 18 in which the illustrative leaflet 19 comprises a first portion and a second portion 58,59 which are each separate components. The individual first and second portion 58,59 include interlocking structure 61 on their respective facing edges which allows the two halves to engage one another after deployment from the delivery member, whereby the joined leaflet 14 is inserted into place with the support housing. The nature of the interlocking structure 61 not particularly critical to the invention, but preferably is configured such that minimal force is necessary to manually insert one portion into another in situ.

One method of unfolding or assembling the valve-closing elements 14 and inserting it into position is shown in FIG. 17. In the illustrative embodiment, the leaflet 19 includes a pair of apertures 66 located in the first and second portions 58,59 with a suture or retention wire 65 traversing each and extending into the lumen of a elongate member 64 where it tethers the two portions to each other. The elongate member 64 is configured for manipulating the leaflet 19 from its folded configuration and inserting it into place with the aid of the retention wire 65 securing the leaflet thereto. The slack within the retention wire 65 can be adjusted depending whether the valve-closing element 14 is being unfolded or positioned. The illustrative method and apparatus can be also used to assemble and manipulate separate elements such as the embodiment of FIG. 18, the first and second portions 58,59 of which being preferably preloaded within the delivery system with the retention wire 65 in place, traversing each unassembled portion.

FIG. 20 depicts and embodiment that avoids some of the challenges associated with expanding or assembling a valve-closing element and engaging it with the valve support housing in the prescribed manner. In the illustrative embodiment, the valve-closing member 14 comprises a free-floating umbrella configuration 67 that functions much as the ball valve 31 element of FIG. 6, whereby there is a seating ring 33 and a proximal retention means 32 to maintain the valve-closing element 14 within a the valve support housing 15. The umbrella 67, which comprises a plurality of foldable plates 57, is compressed into the folded configuration for delivery, whereby it is expanded by some means, such as using appropriately configured intravascular instrumentation, such that the umbrella element 67 assumes the expanded second configuration 55 or balloon to form a functional valve portion 13. To insure a proper seal of the umbrella element 67 against the seating ring 33, the umbrella element 67 can be configured such that when the plates 57 fully expand into the open configuration 55, the plates lock into a fully extended position such that the umbrella element cannot be readily compressed or folded back toward the first configuration.

After the valve-closing element has been converted from the first configuration to the expanded second configuration, it must be positioned into place using an imaging method such a fluoroscopy. FIG. 16 depicts a valve support housing 15 in which the recess/aperture 22 for receiving the appropriate rotational element 21 or pivoting projection/tab of the leaflet 14 includes a pair of imageable markers 62 or indicia to help the clinician locate the recess or aperture 22 under imaging. These can include radiopaque markers made of gold, tungsten, tantalum, platinum, or another suitable high-density material. Such markers can also be located elsewhere on the prosthesis to aid in visualization under radiographic imaging. To further assist the clinician, the projection 21 may further include an imageable marker 63 as well. The imageable markers 62,36 on the valve support housing 15 and valve-closing elements 14 may be configured such that the clinician can distinguish the right or left sides thereof so as not to install the valve-closing element 14 in a backward position.

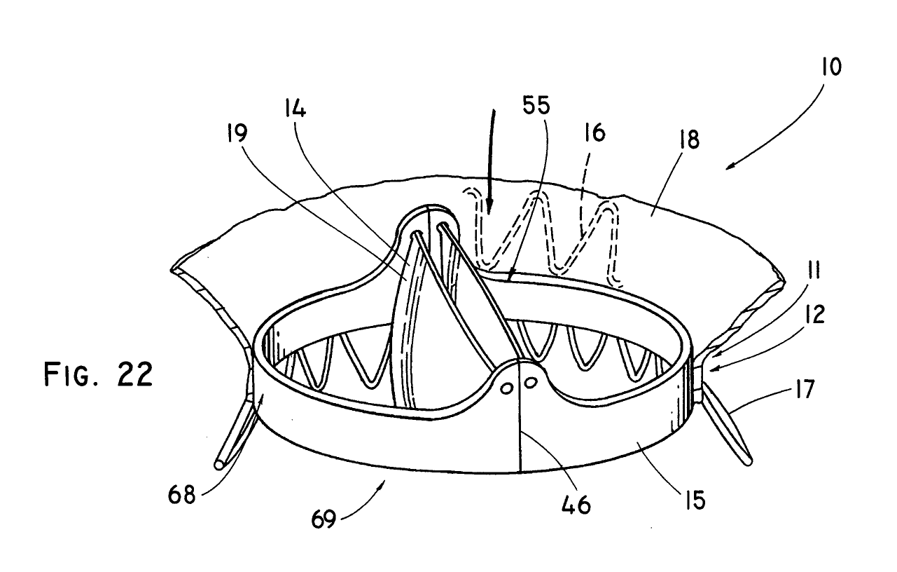

FIG. 21 depicts an embodiments that is configured for use as an artificial aortic or mitral heart valve in which the valve support housing 15 comprises a ring-like structure that is assembled from two or more component pieces (e.g., portions 68 and 69). As in the embodiments of FIGS. 2 and 5, the anchoring portion comprises first and second anchoring support structures 16,17 that include a covering to help form a seal against blood leaking around the valve support housing 15 which receives a pair of valve-closing elements 14, such as the illustrative leaflets 19 which are sufficiently thin such that they can be delivered in a smaller diameter configuration and expanded to their final configuration 55 to form the valve mechanism. To assist in the assembling of the valve housing components 68,69, interlocking structure 72 comprising rare earth or standard magnets, such as that depicted in FIG. 22, may be utilized. In the illustrative embodiment, the projections 73 on adjoining valve support housing components comprise the magnets with their poles aligned such that they can insert into the opposite adjoining face and attach to appropriately configured magnets 74 that are recessed therein to receive the corresponding projections. Preferably, the projection 73 are configured to provide a mechanical locking engagement with the corresponding recess as further assurance that the components 68,69 will not dissemble during the life of the device. Alternatively, the valve support housing 15 can be assembled using radiopaque markers, retention wires, or other techniques that facilitate engaging one valve support housing components to another. The use of magnets is also contemplated for attaching valve-closing elements 14 or separate venous valve support housing components as well. While the illustrative embodiment comprises a two-component 68,69 valve support housing 15, a four-component valve housing would further allow reduction of the size of the delivery system required to delivery the device, insomuch that half of the ring comprising the valve support housing would essentially have the same width as the diameter of the full ring as would thus, have to be straightened somewhat to gain any significant reduction in size. A quarter of the ring comprising the valve support housing 15 could more readily delivered through a smaller delivery member.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention. The invention encompasses embodiments both comprising and consisting of the elements described with reference to the illustrative embodiments. Unless otherwise indicated, all ordinary words and terms used herein shall take their customary meaning as defined in The New Shorter Oxford English Dictionary, 1993 edition. All technical terms shall take on their customary meaning as established by the appropriate technical discipline utilized by those normally skilled in that particular art area. All medical terms shall take their meaning as defined by Stedman's Medical Dictionary, 27^{th} edition.

1. A valve prosthesis for implantation in blood vessel, comprising:
a support structure having a first configuration and a first diameter for delivery through a blood vessel and a second configuration and second diameter for implantation therein, the support structure including a passageway extending therethrough;
the support structure further comprising a valve portion having a first and second end and that includes a valve support housing and one or more valve-closing elements attached thereto and configured to permit blood flowing through the passageway in a first direction and restricting blood flow in a second direction opposite the first direction; and an anchoring portion, the support structure further comprising an anchoring portion attached about the valve portion;
wherein the support structure is configured such that the valve portion is substantially non-self expanding when no longer constrained by the delivery system, while the anchoring portion expands to the second diameter valve portion to engage the walls of the blood vessel and anchors the prosthesis therein.

2. The valve prosthesis of claim 1, wherein the valve portion comprises a deposition of pyrolitic carbon, the deposition of pyrolitic carbon sufficiently covering at least a portion of the valve portion so as to inhibit the formation of thrombus about the blood-contacting surfaces of the valve mechanism.

3. The valve prosthesis of claim 2, wherein the deposition of pyrolitic carbon covers the valve-closing elements.

5. The valve prosthesis of claim 2, wherein the deposition of pyrolitic carbon covering at least the valve-closing elements and the valve support housing.

6. The valve prosthesis of claim 1, wherein the valve housing comprising a tubular-shaped element and the at least one valve-closing element comprises a leaflet attached within the valve housing.

7. The valve prosthesis of claim 6, wherein the at least one valve-closing element comprises a pair of cooperating leaflets configured to pivot about an axis inside the valve housing and contact one another to restrict retrograde flow in the second direction.

8. The valve prosthesis of claim 6, wherein the tubular-shaped element comprises a substantially rigid, non-collapsible configuration having a smooth adluminal surface, the adluminal surface being coated by a portion of the deposition of pyrolitic carbon.

9. The valve prosthesis of FIG. 1, wherein the anchoring portion comprises a covering configured to direct the blood flow through the valve portion.

10. The valve prosthesis of claim 9, wherein the covering comprises a bioremodelable material.

11. The valve prosthesis of claim 9, further comprising a first anchoring support structure attached to the first end of valve portion and a second anchoring support structure attached to the second end of the valve portion.

12. The valve prosthesis of claim 1, wherein the valve mechanism comprises a ball valve having a sealing element and a receiving element such that the sealing element is engageable with the receiving element such that when seated therein, a seal is created against blood flowing therethrough, the valve portion further comprising a constraining mechanism configured to maintain the sealing element within the prosthesis.

13. The valve prosthesis of claim 1, wherein the anchoring portion disposed on the outer surface of the valve portion such that the anchoring portion is expandable to anchor the valve mechanism thereinside.

14. The valve prosthesis of claim 1, wherein the valve support housing comprises a first configuration and a second configuration having a diameter larger than the first configuration, wherein the valve closing elements are configured to be insertable into the valve support housing once the valve support housing is expanded into the second configuration.

15. The valve prosthesis of claim 1, wherein the one or more valve-closing elements comprises a substantially rigid material and are configured to be one of collapsible, foldable, or detachable to assume a first configuration for delivery to the implantation site.

16. The valve prosthesis of claim 1, wherein the valve support housing comprises a substantially rigid material and is configured to be one of collapsible, foldable, or detachable to assume a first configuration for delivery to the implantation site.

17. A valve prosthesis for implantation in a blood vessel comprising:
a support structure having a valve portion attached thereto, wherein the valve portion includes a deposition of pyrolitic carbon on at least a portion of the adluminal surface of the valve mechanism in an effective amount for improving thromboresistance, the support structure further comprising a anchoring portion configured to anchor the valve prosthesis within the implantation site.

18. A valve prosthesis for implantation in a blood vessel comprising:
a valve mechanism having blood-contacting surfaces and a substantially fixed outer diameter comprising the first diameter, the first diameter being sized for insertion into an intravascular sheath for introduction within the blood vessel; and
one or more anchoring elements attached to the valve mechanism, the one or more anchoring elements configured to be expandable to a second diameter that is larger than the first diameter, the second diameter being sufficient for engaging the walls of the blood vessel such that valve prosthesis is anchorable therein.

19. The valve prosthesis of claim 18, wherein the blood-contacting surfaces of the valve mechanism comprises a layer of thromboresistant material.

20. The valve prosthesis of claim 18, wherein the thromboresistant material includes a deposition of pyrolitic carbon.
